(19) United States
(12) Patent Application Publication
(10) Pub. No.: US 2004/0077658 A1
(43) Pub. Date: Apr. 22, 2004

(54) Quinoline-4-carboxamide derivatives as nk-3 and nk-2 receptor antagonists

(76) Inventors: Carlo Farina, Baranzate di Bollate (IT); Giuseppe Giardina, Baranzate di Bollate (IT); Mario Grugni, Baranzate di Bollate (IT); Guy Marguerite Marie Gerard, Rennes (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/416,600

(22) PCT Filed: Nov. 12, 2001
(86) PCT No.: PCT/EP01/13141

(30) Foreign Application Priority Data
Nov. 13, 2000 (GB) 0027701.2

(51) Int. Cl. A61K 31/496; C 07D 4 3/02; C 07D 4 3/14

(52) U.S. Cl. 514/253.06; 544/363; 514/217.05

(57) ABSTRACT

Certain compounds of formula (I) below or a pharmaceutically acceptable salt or hydrate thereof: (I) wherein: R₁ is H or C₁₋₆ alkyl; R₂ is aryl or C₃₋₇ cycloalkyl or heteroaryl; R₃ is H or C₁₋₃ alkyl, optionally substituted by one or more fluorines; R₄ is R₈R₉; R₈ is a single bond, C₁₋₆ alkyl, or aryl; R₉ is H, COO R₁₀, or N R₁₁R₁₂; R₁₀ is H or C₁₋₆ alkyl; R₁₁ and R₁₂ are independently selected from H and C₁₋₆ alkyl; R₅ is branched or linear C₁₋₆ alkyl, C₃₋₇ cycloalkyl, C₄₋₇ cycloalkylalkyl, aryl, or a single or fused ring aromatic heterocyclic group; R⁶ represents H or up to three substituents independently selected from the list consisting of: C₁₋₆ alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluoromethyl, acyloxy, amino or mono- or di-C₁₋₆ alkylamino; R₇ is H or halo; a is 1-6; and any of R₂, R₅, R₈, R₁₀, R₁₁, and R₁₂ may optionally be substituted one or more times by halo, hydroxy, amino, cyano, nitro, carboxy or oxo; a process for preparing such compounds, a pharmaceutical composition comprising such compounds and the use of such compounds and composition in medicine.

The present invention relates to novel compounds, in particular to novel quinoline derivatives, to processes for the preparation of such compounds, to pharmaceutical compositions containing such compounds and to the use of such compounds in medicine.

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol., 13, 23-93).

Selective peptidic NK₃ receptor antagonists are known (Drapeau, 1990 Regul Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J. Physiol., 470, 665-679; Counture et al., 1993, Regul. Peptides, 46, 426-429; Mccarson and Krause, 1994, J. Neurosci., 14 (2), 712-720; Arenas et al. 1991, J. Neurosci., 11, 2332-8). However, the peptide-like nature of the known antagonists makes them likely to be too labile from a metabolic point of view to serve as practical therapeutic agents.

International Patent Application, Publication number WO 00/31037 discloses certain compounds stated to be non-peptide NK-3 antagonists and also to have NK-2 antagonist activity. These compounds are disclosed to be of potential use in the prevention and treatment of a wide variety of clinical conditions which are characterised by overstimulation of the Tachykinin receptors, in particular NK-3 and NK-2.

We have now discovered a further novel class of potent non-peptide NK-3 antagonists some of which fall within the generic scope of WO 00/31037. The new compounds are also far more stable from a metabolic point of view than the known peptidic NK-3 receptor antagonists and are of potential therapeutic utility. The new compounds also have good NK-2 antagonist activity and are therefore considered to be of potential use in the prevention and treatment of a wide variety of clinical conditions which are characterised by overstimulation of the Tachykinin receptors, in particular NK-3 and NK-2.

These conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies (hereinafter referred to as the ‘Primary Conditions’).

Certain of these compounds also show CNS activity and hence are considered to be of particular use in the treatment of disorders of the central nervous system such as anxiety, depression, psychosis and schizophrenia, neurodegenerative disorders such as AIDS related dementia, senile dementia of the Alzheimer type, Alzheimer's disease, Down's syndrome, Huntington's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilation and vasospastic diseases such as angina, migraine and Reynaud's disease and pain or nociception, for example, that is attributable to or associated with any of the foregoing conditions especially the transmission of pain in migraine, (hereinafter referred to as the ‘Secondary Conditions’).

The compounds of formula (I) are also considered to be useful as diagnostic tools for assessing the degree to which neurokinin-3 and neurokinin-2 receptor activity (normal, overactivity or underactivity) is implicated in a patient's symptoms.

According to the present invention, there is provided a compound of formula (I) below or a pharmaceutically acceptable salt or hydrate thereof:

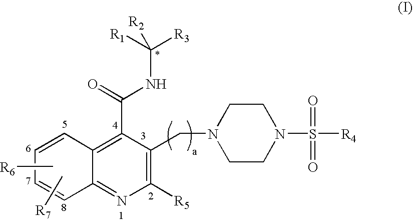

(I)

wherein:

R₁ is H or C₁₋₆ alkyl;

R₂ is aryl or C₃₋₇ cycloalkyl or heteroaryl;

R₃ is H or C₁₋₃ alkyl, optionally substituted by one or more fluorines;

R₄ is R₈R₉;

R₈ is a single bond, C₁₋₆ alkyl, or aryl;

R₉ is H, COO R₁₀, or N R₁₁R₁₂;

R₁₀ is H or C₁₋₆ alkyl;

R₁₁ and R₁₂ are independently selected from H and C₁₋₆ alkyl, or are joined to form a 5-7 membered heterocyclic ring;

R₅ is branched or linear C₁₋₆ alkyl, C₃₋₇ cycloallyl, C₄₇ cycloalkylalkyl, aryl, or a single or fused ring aromatic heterocyclic group;

R₆ represents H or up to three substituents independently selected from the list consisting of: C₁₋₆ alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluoromethyl, acyloxy, amino or mono- or di-C₁₋₆ alkylamino;

R₇ is H or halo;

a is 1-6; and

any of R₂, R₅, R₈, R₁₀, R₁₁ and R₁₂ may optionally be substituted one or more times by halo, hydroxy, amino, cyano, nitro, carboxy or oxo; subject to the proviso that said compound is not a compound of formula (I) wherein R₇ represents H, R₆ represents H, R₅ represents phenyl, a is 1, and R₁, R₂, R₃ and R4 are one of the following combinations:

| 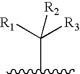 | R₄ |
|---|---|
| 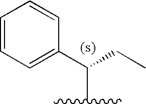 | —NH₂ |
| 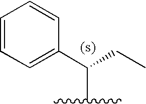 | 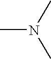 |

Advantageously, R₃ represents methyl or ethyl or iso-propyl.

Suitably, R₂ represents phenyl or cyclohexyl.

Preferably, R₁ is hydrogen.

Suitably, R₅ is unsubstituted phenyl.

Optionally, each of R₆ and R₇ may represent hydrogen.

In preferred embodiments, a is 1-3. Most preferably, a is 1.

Advantageously, R₈ is methyl, ethyl or phenyl. In especially preferred embodiments, R₈ is methyl. In other preferred embodiments, R₈ is phenyl.

In some embodiments, R₉ is H.

In other embodiments, R₉ is COOR₁₀ and R₁₀ is H or methyl or ethyl.

In yet other embodiments, R₉ is NR₁₁R₁₂, and R₁₁ and/or R₁₂ is methyl, ethyl or propyl. Suitably, each of R₁₁ and R₁₂ is the same one of ethyl or propyl.

In particularly preferred embodiments, a is 1, R₆ is H, R₁ is H, R₅ is unsubstituted phenyl, R₇ is hydrogen, and R₂, R₃ and R₄ are selected from the following combinations:

| R₂ | R₃ | R₄ |
|---|---|---|
| phenyl | isopropyl | 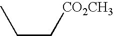 |
| phenyl | isopropyl | 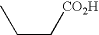 |
| phenyl | ethyl | 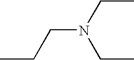 |
| cyclohexyl | methyl | —CH₃ |
| cyclohexyl | methyl | 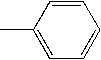 |

The compounds of formula (I) may have at least one asymmetric centre—for example the carbon atom labelled with an asterisk (*) in the compound of formula (I)— and therefore may exist in more than one stereoisomeric form. The invention extends to all such stereoisomeric forms and to mixtures thereof, including racemates. In particular, the invention includes compounds wherein the asterisked carbon atom in formula (I) has the stereochemistry shown in formula (Ia):

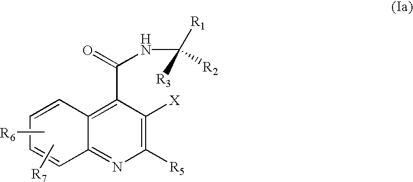

wherein R₁, R₂, R₃, R₅, R₆, and R₇ are as defined in relation to formula (I), and X represents the moiety

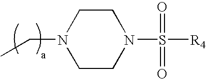

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, fumaric, salicylic, citric, lactic, mandelic, tartaic, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkcylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable solvates are pharmaceutically acceptable solvates.

Suitable pharmaceutically acceptable solvates include hydrates.

The term ‘alkyl’ (unless specified to the contrary) when used alone or when forming part of other groups (such as the ‘alkoxy’ group) includes straight- or branched-chain alkyl groups containing 1 to 12 carbon atoms, suitably 1 to 6 carbon atoms, examples include methyl, ethyl, n-propyl, isopropyl, n-butyl, isobutyl or tert-butyl group.

The term ‘carbocylic’ refers to cycloalkyl and aryl rings.

The term ‘cycloalkyl’ includes groups having 3 to 12, suitably 4 to 6 ring carbon atoms.

The term ‘aryl’ includes phenyl and naphthyl, preferably phenyl which unless specified to the contrary optionally comprise up to five, preferably up to three substituents selected from halogen, allyl, phenyl, alkoxy, haloalkyl, hydroxyalkyl, hydroxy, amino, nitro, cyano, carboxy, alkoxycarbonyl, alkoxycarbonylalkyl, alkylcarbonyloxy, or alkylcarbonyl groups.

The term ‘aromatic heterocyclic group’ includes groups comprising aromatic heterocyclic rings containing from 5 to 12 ring atoms, suitably 5 or 6, and comprising up to four hetero-atoms in the or each ring selected from S, O or N.

Composite terms such as ‘alkylcarboxy’, ‘cycloalkylalkyl’ and so forth refer to components of a compound which include two interlinked groups, with the group named latterly in the term being the linking group, so that ‘alkylcarboxy’ means (alkyl)-COO— whilst ‘cycloalkylalkyl’ means (cycloalkyl)-(alkyl)-.

Unless specified to the contrary, suitable substituents for any heterocyclic group includes up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

When used herein the term “halogen” or “halo” refers to fluorine, chlorine, bromine and iodine, preferably fluorine, chlorine or bromine.

It will be understood that unless specified to the contrary, groups and substituents defined herein are unsubstituted.

When used herein the term “acyl” includes residues of acids, in particular a residue of a carboxylic acid such as an alkyl- or aryl- carbonyl group.

The invention also provides a process for the preparation of a compound of formula (I), or a salt thereof and/or a solvate thereof, which process comprises reacting a compound of formula (II) or an active derivative thereof:

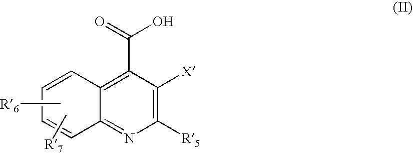

wherein R′₆, R′₇, R′₅ and X′ are R₆, R₇, R₅ and X respectively as hereinbefore defined in relation to formula (I) or (Ia), or a group convertible to R₆, R₇, R₅ and X respectively; with a compound of formula (III):

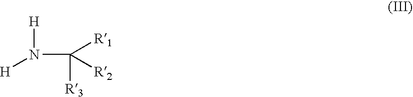

wherein R₁′, R₂ ′, and R₃′ are R₁, R₂, and R₃ as defined for formula (I) or a group or atom convertible to R₁, R₂, and R₃ respectively; to form a compound of formula (Ib):

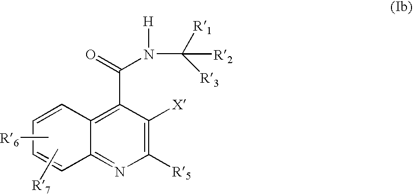

wherein R′₁, R′₂, R′₃, X′, R′₅, R′₆ and R′₇ are as defined above, and thereafter carrying out one or more of the following optional steps:

(i) converting any one of R′₁, R′₂, R′₃, X′, R′₅, R′₆ and R′₇ to R₁, R₂, R₃, X, R₅, R₆ and R₇ respectively as required, to obtain a compound of formula (I); and

(ii) converting a compound of formula (I) into another compound of formula (I); and

(iii) preparing a salt of the compound of formula (I) and/or a solvate thereof.

Suitable groups convertible into other groups include protected forms of said groups.

Suitably R′₁, R′₂, R′₃, X′, R′₅, R′₆ and R′₇ each represents R₁, R₂, R₃, X, R₅, R₆ and R₇ respectively or a protected form thereof or a group convertible thereto.

In preferred embodiments, X′ represents

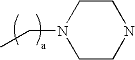

and said process for preparing a compound of formula (I) further includes the step of reacting said compound (Ib) with a compound of formula

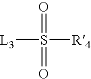

wherein L₃ represents a leaving group for example halogen, preferably chlorine or bromine, and R′₄ represents R₄ or a protected form thereof or a group convertible thereto.

It is favoured if the compound of formula (II) is present as an active derivative.

A suitable active derivative of a compound of formula (II) is a transient activated form of the compound of formula (II) or a derivative wherein the carboxy group of the compound of formula (II) has been replaced by a different group or atom, for example by an acyl halide, preferably a chloride, or an acylazide or a carboxylic acid anhydride.

Other suitable active derivatives include: a mixed anhydride formed between the carboxyl moiety of the compound of formula (II) and an alkyl chloroformate; an activated ester, such as a cyanomethyl ester, thiophenyl ester, p-nitrophenyl ester, p-nitrothiophenyl ester, 2,4,6-trichlorophenyl ester, pentachlorophenyl ester, pentafluorophenyl ester, N-hydroxy-phtalimido ester, N-hydroxypiperidine ester, N-hydroxysuccinimide ester, N-hydroxy benzotriazole ester; alternatively, the carboxy group of the compound of formula (II) may be activated using a carbodiimide or N,N′-carbonyldiimidazole.

The reaction between the compound of formula (II) or the active derivative thereof and the compound of formula (III) is carried out under the appropriate conventional conditions for the particular compounds chosen. Generally, when the compound of formula (II) is present as an active derivative the reaction is carried out using the same solvent and conditions as used to prepare the active derivative, preferably the active derivative is prepared in situ prior to forming the compound of formula (Ib) and thereafter the compound of formula (I) or a salt thereof and/or a solvate thereof is prepared.

For example, the reaction between an active derivative of the compound of formula (II) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (II) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofuran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 1 shown below:

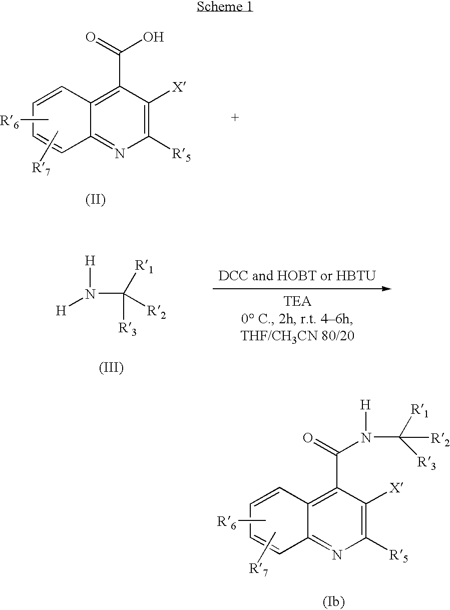

wherein R′₁, R′₂, R′₃, X′, R′₅, R′₆ and R′₇ are as defined above.

It will be appreciated that a compound of formula (Ib) may be converted to a compound of formula (I), or one compound of formula (I) may be converted to another compound of formula (I) by interconversion of suitable substituents. Thus, certain compounds of formula (I) and (Ib) are useful intermediates in forming other compounds of the present invention.

Accordingly, in a further aspect the invention provides a process for preparing a compound of formula (I), or a salt thereof and/or a solvate thereof, which process comprises converting a compound of the above defined formula (Ib) wherein at least one of R′₁, R′₂, R′₃, X′, R′₅, R′₆ and R′₇ is not R₁, R₂, R₃, X, R₅, R₆ or R₇ respectively, thereby to provide a compound of formula (I); and thereafter, as required, carrying out one or more of the following optional steps:

(i) converting a compound of formula (I) into another compound of formula (I); and

(ii) preparing a salt of the compound of formula (I) and/or a solvate thereof.

Suitably, in the compound of formula (Ib) the variables R′₁, R′₂, R′₃, X′, R′₅, R′₆ and R′₇ are R₁, R₂, R₃, X, R₅, R₆ and R₇ respectively or they are protected forms thereof.

The above mentioned conversions, protections and deprotections are carried out using the appropriate conventional reagents and conditions and are further discussed below.

Where X′ represents

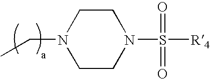

a compound of formula (II) or the corresponding alkyl (such as methyl or ethyl) ester, may conveniently be prepared by reacting a compound of formula (IV) or the corresponding alkyl (such as methyl or ethyl) ester:

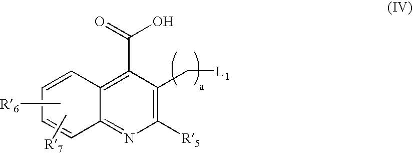

wherein R′₆, R′₇, R′₅ and a are as defined above and L₁ represents a halogen atom such as a bromine atom, with a compound of formula (V):

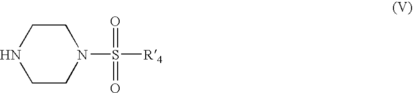

wherein R′₄ is R₄ as defined in relation to formula (I) or a protected form thereof.

Suitably, R′₄ is R₄.

Alternatively, where X′ represents

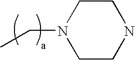

a compound of formula (II) or the corresponding alkyl (such as methyl or ethyl) ester, may conveniently be prepared by reacting a compound of formula (IV) or the corresponding alkyl (such as methyl or ethyl) ester:

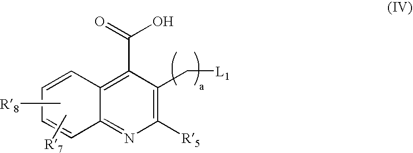

wherein R′₆, R′₇, R′₅ and a are as defined above and L₁ represents a halogen atom such as a bromine atom, with a compound of formula (V):

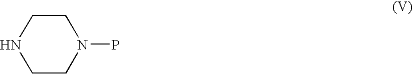

wherein P is an amine protective group, for example fmoc or benzyl, preferably fmoc, and thereafter removing said protective group. The protective group may be removed by standard methods described in the literature, for example the fmoc residue may be split by action of piperidine at room temperature in a solvent like acetonitrile.

Suitably, reaction between the compounds of formulae (IV) or the corresponding alkyl (such as methyl or ethyl) ester and (V) is carried out under conventional amination conditions, for example when L₁ is a bromine atom then the reaction is conveniently carried out in an aprotic solvent, such as tetrahydrofuran or dimethylformamide at any temperature providing a suitable rate of formation of the required product, usually at ambient temperature; preferably the reaction is carried out in the presence of triethylamine (TEA) or K₂CO₃.

A compound of formula (IV) or the corresponding alkyl (such as methyl or ethyl) ester is prepared by appropriate halogenation of a compound of formula (VI) or the corresponding alkyl (such as methyl or ethyl) ester:

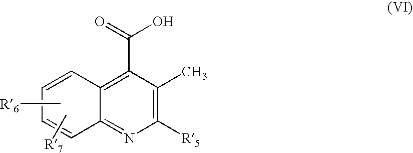

wherein R′₆, R′₇ and R′₅ are as defined above in relation to formula (II).

Suitable halogenation reagents are conventional reagents depending upon the nature of the halogen atom required, for example when L₁ is bromine a preferred halogenation reagent is N-bromosuccinimide (NBS).

The halogenation of the compound of formula (VI) or the corresponding alkyl (such as methyl or ethyl) ester is carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

In the case in which the corresponding alkyl (such as methyl or ethyl) ester of compounds (VI), (IV) and (II) are utilised, an hydrolysis to compound (II) is required before conversion to compound (Ib) in Scheme 1. Such hydrolysis can be carried out under acidic conditions, such 10-36% hydrochloric acid at a temperature in the range between 30 and 100 ° C.

A compound of formula (II) wherein X′ represents

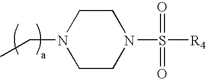

may conveniently be prepared by reacting a compound of formula (VII):

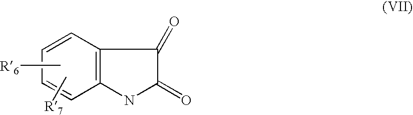

wherein R′₆ and R′₇ are as defined in relation to formula (II), with a compound of formula (VIII):

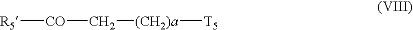

wherein R′₅ is as defined in relation to formula (II), and T₅ is a group

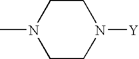

where Y is a protecting group such as a benzyl group, particularly a protecting group which is stable in basic conditions such as a terbutoxycarbonyl group, or a group SO₂R₄ as defined in relation to formula (I) or a protected form thereof or a group convertible thereto, and a is an integer in the range of 1 to 6; and thereafter as required removing any protecting group, for example by dehydrogenation, and/or converting any group T₅ to

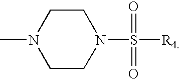

The reaction between the compounds of formula (VII) and (VIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

Protected forms of

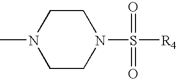

will vary according to the particular nature of the group being protected but will be chosen in accordance with normal chemical practice.

Groups convertible to

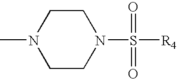

include groups dictated by conventional chemical practice to be required and to be appropriate, depending upon the specific nature of the

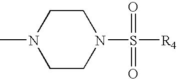

under consideration.

Suitable deprotection methods for deprotecting protected forms of

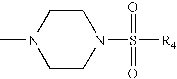

and conversion methods for converting T₅ to

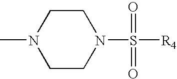

will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

A compound of formula (VIII) is prepared from a compound of formula (ID):

R₅′—CO—CH₂—(CH₂)ₐ—OH (IX)

wherein R′₅ is as defined in relation to formula (II) and a is as defined in relation to formula (VIII), by first halogenating, preferably brominating, or mesylating the compound of formula (IX) and thereafter reacting the halogenation or mesylafion product so formed with a compound capable of forming a group T₅ so as to provide the required compound of formula (VII).

When T₅ is a group

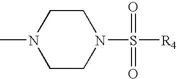

a compound capable of forming a group T₅ is a compound of the above defined formula (V).

The halogenation of the compound of formula (IX) is suitably carried out using a conventional halogenation reagent. Mesylation is conveniently carried out using mesyl chloride in an inert solvent such as methylene dichloride, at a temperature below room temperature, such as 0° C., preferably in the presence of triethylamine.

The reaction conditions between the compound of formula (IX) and the compound capable of forming a group T₅ will be those conventional conditions dictated by the specific nature of the reactants, for example when the T₅ required is a group

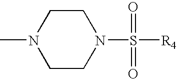

and the required compound capable of forming a group T₅ is a compound of the above defined formula (V), then the reaction between the halogenationor mesylation product of the compound of formula (IX) and the compound of formula (V) is carried out under analogous conditions to those described for the reaction between the compounds of formulae (IV) and (V). Other compounds capable of forming a group T₅ will depend upon the particular nature of T₅, but will be those appropriate compounds dictated by conventional chemical practice with reference to standard texts such as Chemistry of the Amino Group, Patais (Ed.), Interscience, New York 1968; and Advanced Organic Chemistry, March J, John Wiley & Sons, New York, 1992.

A compound of formula (IX) may be prepared by reacting a compound of formula (X):

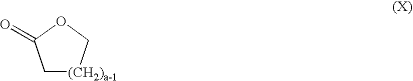

wherein a is as defined in relation to formula (VIII), with a lithium salt of formula (XI):

R′₅ Li (XI)

wherein R′₅ is as defined in relation to formula (II).

The reaction between the compounds of formulae (X) and (XI) can be carried out in an aprotic solvent, such as diethyl-ether at any temperature providing a suitable rate of formation of the required product, usually at a low temperature such as in the range of −10° C. to −30° C., for example −20° C.

The compounds of formula (III) are known commercially available compounds or they can be prepared from known compounds by known methods, or methods analogous to those used to prepare known compounds, for example the methods described in Liebigs Ann. der Chemie, (1936), 523, 199.

A chiral compound of formula (III) wherein R₂ is a C₅ or C₇ cycloalkyl group, R₃ is methyl and R₁ is H are described in J. Org. Chem. (1996), 61 (12), 4130-4135. A chiral compound of formula (III) wherein R₂ is phenyl, R₃ is isopropyl and R₁ is H is a known compound described in for example Tetrahedron Lett. (1994), 35(22), 3745-6.

The compounds of formula (V) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

The compounds of formula (VII) are known compounds or they are prepared according to methods used to prepare known compounds for example those disclosed in J. Org. Chem. 21, 171 (1955); J. Org. Chem. 21, 169 (1955).

The compounds of formula (X) and (XI) are known compounds or they are prepared according to methods used to prepare known compounds for example those disclosed by Krow G. R. in Organic Reactions, Vol 43, page 251, John Wiley & Sons Inc.1994 (for the compounds of formula (X)) and Organometallics in Synthesis, Schlosser M.(Ed), John Wiley & Sons Inc.1994 (for the compounds of formula (XI)).

As hereinbefore mentioned, the compounds of formula (I) may exist in more than one stereoisomeric form—and the process of the invention may produce racemates as well as enantiomerically pure forms. Accordingly, a pure enantiomer of a compound of formula (I) is obtained by reacting a compound of the above defined formula (II) with an appropriate enantiomerically pure primary amine of formula (IIIa) or (IIIc):

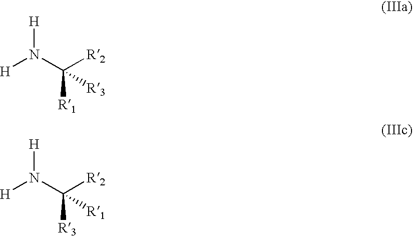

wherein R′₁, R′₂ and R′₃ are as defined above, to obtain a compound of formula (I′a) or (I′c):

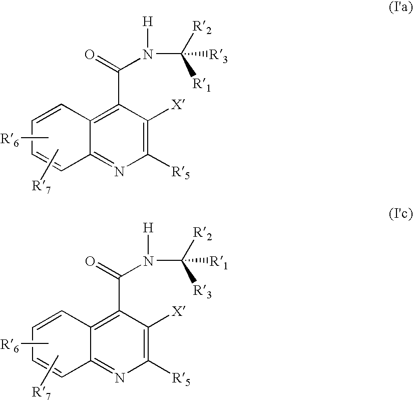

wherein R′₁, R′₂, R′₃, X′, R′₅, R′₆, and R′₇ are as defined above.

Compounds of formula (I′a) or (I′c) may subsequently be converted to compounds of formula (Ia) or (Ic) by the methods of conversion mentioned before:

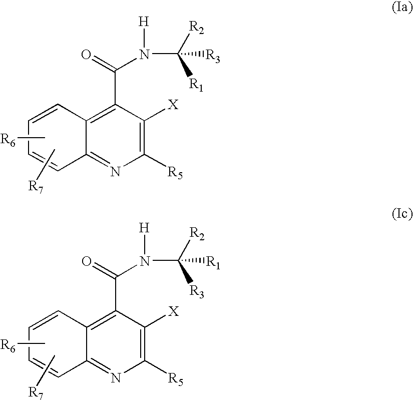

wherein R₁, R₂, R₃, X, R₅, R₆, and R₇ are as defined above.

Suitably, in the above mentioned compounds of formulae (Ia), (Ic), (I′a), (I′c), (IIIa) and (IIIc) R₁ represents hydrogen.

An alternative method for separating optical isomers is to use conventional, fractional separation methods in particular fractional crystallization methods. Thus, a pure enantiomer of a compound of formula (I) is obtained by fractional crystallisation of a diastereomeric salt formed by reaction of the racemic compound of formula (I) with an optically active strong acid resolving agent, such as camphosulphonic acid,, tartaric acid, O,O′-di-p-toluoyltartaric acid or mandelic acid, in an appropriate alcoholic solvent, such as ethanol or methanol, or in a ketonic solvent, such as acetone. The salt formation process should be conducted at a temperature between 20° C. and 80° C., preferably at 50° C.

A suitable conversion of one compound of formula (I) into a further compound of formula (I) involves converting one group X into another group X by for example:

(i) converting a ketal into a ketone, by such as mild acidic hydrolysis, using for example dilute hydrochloric acid;

(ii) reducing a ketone to a hydroxyl group by use of a borohydride reducing agent;

(iii) converting a carboxylic ester group into a carboxyl group using basic hydrolysis; and/or

(iv) reducing a carboxylic ester group to a hydroxymethyl group, by use of a borohydride reducing agent.

As indicated above, where necessary, the conversion of any group R′₁, R′₂, R′₃, X′, R′₅, R′₆, and R′₇ into R₁, R₂, R₃, X, R₅, R₆, and R₇ which as stated above are usually protected forms of R₁, R₂, R₃, X, R₅, R₆, or R₇ may be carried out using appropriate conventional conditions such as the appropriate deprotection procedure.

It will be appreciated that in any of the above mentioned reactions any reactive group in the substrate molecule may be protected and deprotected according to conventional chemical practice, for example as described by Greene, T. W. and Wuts, P. G. M. Protective Groups in Organic Synthesis, John Wiley & Sons Inc. New York, 1991 (Second Edt.) or in Kociensid, P. J. Protecting groups. George Thieme Verlag, New York, 1994.

Suitable protecting groups in any of the above mentioned reactions are those used conventionally in the art. Thus, for example suitable hydroxyl protecting groups include benzyl or trialkylsilyl groups.

The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using catalytic hydrogenation or a mild ether cleavage reagent such as trimethylsilyl iodide or boron tribromide.

As indicated above, the compounds of formula (I) have useful pharmaceutical properties.

Accordingly the present invention also provides a compound of formula (I), or a pharmaceutically acceptable salt or solvate thereof, for use as an active therapeutic substance.

In particular, the present invention also provides a compound of formula (I), or a pharmaceutically acceptable salt or solvate thereof, for the treatment or prophylaxis of the Primary and Secondary Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions.

As mentioned above the Primary conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such.as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies.

As mentioned above, the Secondary conditions include disorders of the central nervous system such as anxiety, depression, psychosis and schizophrenia; neurodegenerative disorders such as AIDS related dementia, senile dementia of the Alzheimer type, Alzheimer's disease, Down's syndrome, Huntington's disease, Parkinson's disease, movement disorders and convulsive disorders (for example epilepsy); demyelinating diseases such as multiple. sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilation and vasospastic diseases such as angina, migraine and Reynaud's disease and pain or nociception, for example, that is attributable to or associated with any of the foregoing conditions especially the transmission of pain in migraine.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery too the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands, [¹²⁵I]-[Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophiys. Res. Commun., 198(3), 967-972).

The binding assays utilized allow the determination of the concentration of the individual compound required to reduce by 50% the [¹²⁵I]-[Me-Phe⁷]-NKB and [³H]-Senktide specific binding to NK₃ receptor in equilibrium conditions (IC50).

Binding assays provide for each compound tested a mean IC₅₀ value of 2-5 separate experiments performed in duplicate or triplicate. The most potent compounds of the present invention show IC₅₀ values in the range 0.1-1000 nM. The NK₃-antagonist activity of the compounds of the present invention is determined by their ability to inhibit senktide-induced contraction of the guinea-pig ileum (Maggi et al, 1990, Br. J Pharmacol., 101, 996-1000) and rabbit isolated iris sphincter muscle (Hall et al., 1991, Eur. J. Pharmacol., 199, 9-14) and human NK₃ receptors-mediated Ca⁺⁺ mobilization (Mochizuki et al, 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilization induced by the agonist NKB. In this assay, the compounds of the present invention behave as antagonists.

The activity of the compounds of the present invention, as NK-2 ligands, is determined by their ability to inhibit the binding of the radiolabelled NK-2 ligands, [¹²⁵I]-NKA or [³H]-NKA, to human NK-2 receptors (Aharony et al, 1992, Neuropeptide, 23, 121-130).

The binding assays utilized allow the determination of the concentration of the individual compound required to reduce by 50% the [¹²⁵I]-NKA and [³H]-NKA specific binding to NK2 receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.5-1000 nM, such as 1-1000 nM. The NK-2-antagonist activity of the compounds of the present invention is determined by their ability to inhibit human NK-2 receptor-mediated Ca⁺⁺ mobilization (Mochizuki et al, 1994, J Biol. Chem., 269,9651-9658). Human receptor functional assay allows the determination of the concentration of the individual compound required to reduce by 50% (IC₅₀ values) the Ca⁺⁺ mobilization induced by the agonist NKA. In this assay, the compounds of the present invention behave as antagonists.

The therapeutic potential of the compounds of the present invention in treating the conditions can be assessed using rodent disease models.

As stated above, the compounds of formula (I) are also considered to be useful as diagnostic tools. Accordingly, the invention includes a compound of formula (I) for use as diagnostic tools for assessing the degree to which neurokinin-3 and neurokinin-2 receptor activity (normal, overactivity or underactivity) is implicated in a patient's symptoms. Such use comprises the use of a compound of formula (I) as an antagonist of said activity, for example including but not restricted to tachykini agonist-induced inositol phosphate turnover or electrophysiological activation, of a cell sample obtained from a patient. Comparison of such activity in the presence or absence of a compound of formula (I), will disclose the degree of NK-3 and NK-2 receptor involvement in the mediation of agonist effects in that tissue.

The following Descriptions illustrate the preparation of the intermediates, whereas the following Examples illustrate the preparation of the compounds of the invention.

### DESCRIPTIONS AND EXAMPLES

Description A: 3-Methyl-2-phenyl-quinoline4-carboxylic acid methyl ester

30 g (114 mmol) of 3-methyl-2-phenyl-quinoline-4-carboxylic acid (CAS [43071-45-0]) were suspended in 250 ml of dry CH₂Cl₂; 20 ml (230 mmol) of oxalyl chloride dissolved in 120 ml of CH₂Cl₂ were added dropwise and the reaction mixture was stirred at room temperature for 30 min. Two drops of N,N-dimethylformamide (DMF) were added and the reaction was stirred for additional 30 min. The solvent was evaporated in vacuo to dryness, the residue was taken up with 100 ml of CH₂Cl₂ and 100 ml of MeOH, dissolved in 400 ml of CH₂Cl₂, were added dropwise. After stirring for 18 h, the solvent was evaporated in vacuo to dryness, the residue was taken up with CH₂Cl₂ and washed with 1% NaHCO₃; the organic layer was dried over Na₂SO₄, filtered and evaporated in vacuo to dryness to yield 31.6 g of the title compound as a solid, which was used in the following reaction without further purification.

C₁₈H₁₅NO₂

MW277.31

MP=73-75° C.

IR (KBr) 3441, 3051, 2954, 1731, 1582, 1556 cm⁻¹.

Description B: 3-Bromomethyl-2-phenyl-quinofine-4-carboxylic Acid Methyl Ester

10 g (36 mmol) of 3-methyl-2-phenyl-quinoline4-carboxylic acid methyl ester (compound of Description A) were dissolved in 500 ml of CH₃CN; 13 g (72 mmol) of N-bromosuccinimide were added and the reaction mixture was heated to reflux. After adding 1 g (4.1 mmol) of dibenzoylperoxide, the reaction was refluxed for 24 h; then additional 4 g (22.5 mmol) of N-bromosuccinimide and 0.5 g (2.0 mmol) of dibenzoylperoxide were added and the reaction was refluxed for 4 h. The solvent was evaporated in vacuo to dryness to yield 26.1 g of crude methyl 3-bromomethyl-2-phenylquinoline-4-carboxylate (theorical amount, 12.8 g) which was used in the following reaction without further purification.

C₁₈H₁₄BrNO₂

MW=356.23

Description 1: 3-(4-Fmoc-piperazin-1-ylmethyl)-2-phenyl-quinoline-4-carboxylic Acid Methyl Ester

6.6 g (18.5 mmol) of crude 3-bromomethyl-2-phenyl-quinoline-4-carboxylic acid methyl ester (compound of Description B) were dissolved, under nitrogen atmosphere, in 100 ml of dry ThF. The solution was cooled to 10 ° C. and 6.8 g (20 mmol) of Fmoc piperazine, dissolved in 50 ml of ITF, were added dropwise. The reaction mixture was allowed to warm to room temperature and stirred overnight. Salts were filtered off and the filtrate was evaporated in vacuo to dryness, taken up with 2 N HCl and washed with EtOAc; the aqueous layer was basified with 10% NaOH and extracted with CH₂Cl₂. The organic layer was dried over Na₂SO₄, filtered and evaporated in vacuo to dryness to obtain a crde material. Flash chromatography on silica gel afforded 7.5 g (yield: 69%) of the title compound.

C₃₇H₃₃N₃O₄

MW=583.68

¹H NMR δ(DMSOd₆): 1.99(4H); 3.10(4H); 3.62(2H); 3.97(3H); 4.20(1H); 4.42(2H); 7.18-7.40(4Har); 7.45-7.92(12Har); 8.09(1Har)ppm.

Description 2: 3-(4-Fmoc-piperazin-1-ylmethyl)-2-phenyl-quinoline-4-carboxylic acid hydrochloride

7.5 g (13 mmol) of the ester of Description 1 were dissolved in 150 ml of 6 N HCl and refluxed for 1 h. Evaporation to dryness afforded 9.5 g of crude title compound, which was used in the following reaction without further purification.

C₃₆H₃₁N₃O₄.HCl

MW=606.12

H NMR δ(DMSOd₆): 2.50(4H); 3.32(4H); 4.22(2H); 4.23(1H); 4.35(2H); 6.50(1Hexch with D₂O); 7.22-7.88(14Har); 7.98(1Har); 8.17(2Har)ppm.

DESCRIPTION 3: 3-(4-Fmoc-piperazin-1-ylmethyl)-2-phenyl-quinoline-4-carboxylic acid ((S)-1-phenyl-propyl)-amide

5.35 g (8.3 mmol) of crude acid of Description 2 were dissolved in 100 ml of dry THF; 1.7 ml (12.5 mmol) of triethylamine (TEA) and 4.1 g (10.79 mmol) of O-benzotriazol-1-yl-N, N,N′,N′-tetramethyluroniumhexafluoro-phosphate (HBTU) were added and the reaction mixture was cooled at 0° C. 1.7 ml (12.5 mmol) of (S)-1-phenyl-propylamine, dissolved in 40 ml of dry CH₂Cl₂, were added dropwise and the reaction mixture was stirred at room temperature for 24 h and at 50 ° C. for 2 h. The solvent was evaporated in vacuo to dryness and the residue was taken up with EtOAc and washed with H₂O, 1 N NaOH and brine, dried over Na₂SO₄ and evaporated to dryness . Flash chromatography on silica gel afforded 3.2 g (56%) of the title compound.

C₄₅H₄₂N₄O₃

MW=686.86

¹H NMR δ(DMSOd₆): 0.94(3H); 1.40-2.18(6H); 2.57-3.13(4H); 3.50(2H); 4.21(1H); 4.34(2H); 5.08(1H); 7.09-7.98(21Har); 8.03(1Har): 9.12(1Hexch with D ₂O)ppm.

Description 4: 3-(4-Fmoc-piperazin--ylmethyl)-2-phenyl-quinoline-4-carboxylic acid ((S)-1-cyclohexyl-ethyl)-amide

4.75 g (8.3 mmol) of crude acid of Description 2 were condensed on 1.65 ml (11 mmol) of (S)-1-cyclohexyl-ethylamine following the procedure of Description 3 affording, after flash chromatography on silica gel, 2.2 g (yield 43.9%) of the title compound.

C₄₄H₄₆N₄O₃

MW=678.87

¹H NMR δ(DMSOd₆): 0.95(3H); 1.68-4.00(21H); 2.60(3H); 5.08(1H); 7.22-8.24(13Har); 8.11(1Har); 9.32(1Hexch with D2O); 10.82(2Hexch with D₂O)ppm.

Description 5: 2-Phenyl-3-piperazin-1-ylmethyl-quinoline-4-carboxylic acid ((S)-1-phenyl-propyl)-amide

2.75 g (41 mmol) of the Fmoc protected derivative of Description 3 was reacted with 1.0 ml of piperidine in 100 ml acetonitrile, at room temperature for one night. The reaction mixture is concentrated to dryness and the residue was purified by flash chromatography on silicagel, affording 1.14 g (yield 60%) of the title compound.

C₃₀H₃₂N₄O

MW=464.61

¹H NMR δ(DMSOd₆): 0.94(3H); 1.57-2.08(6H); 2.31(4H); 3.36(2H and 1Hexch with D₂O); 5.07(1H); 7.13-7.94(13Har); 8.01(1Har); 9.17(1Hexch with D₂O)ppm. DESCRIPTION 6: 3-(4-Fmoc-piperazin-1-ylmethyl)-2-phenyl-quinoline-4-carboxylic acid ((S)-2-methyl-1-phenyl-propyl)-amide

6.95 g (10.8 mmol) of crude acid of Description 2 were condensed on 2 g (13.5 mmol) of (S)-2-methyl-1-phenyl propylamine following the procedure of Description 3 affording, after flash chromatography on silica gel, 5.4 g (yield 71%) of the title compound.

C₄₆H₄₄N₄O₃

MW=700.86

¹H NMR δ(CDCl₃): 0.96(3H); 1.18(3H); 1.56-2.98(4H); 2.28(1H); 3.04(4H); 3.53(2H); 4.20(1H); 4.35(2H); 5.17(1H); 7.18-7.63(18Har); 7.74(3Har); 7.97(1Hexch with D₂O); 8.14(1Har)ppm.

Description 7: 2-Phenyl-3-piperazin-1-ylmethyl-quinoline-4-carboxylic acid ((S)-2-methyl-1-phenyl-propyl)-amide

5.4 g (7.7 mmol) of the Fmoc derivative of Description 6 was reacted with 1.25 ml of piperidine in 200 ml acetonitrile, at room temperature for one night. The reaction mixture was concentrated to dryness and the residue was purified by flash chromatography on silicagel (eluant: CH₂Cl₂/CH₃OH/NH₄OH; 90/10/2), affording 2.55. g (yield 69.3%) of the title compound.

C₃₁H₃₄N₄O

MW=478.64

¹H NMR δ(DMSOd₆): 0.79(3H); 1.06(3H); 1.49-2.55(91); 3.45(2H and 1Hexch with D₂O); 4.88(1H); 7.12-8.10(14Har); 9.16(1Hexch with D₂O)ppm.

Description 8: 2-Phenyl-3-piperazin-1-ylmethyl-quinoline4-carboxylic acid ((S)-1-cyclohexyl-ethyl)-amide

Synthesised starting from the compound of Description 4 and following the procedure of Description 5.

C₂₉H₃₆N₄O

MW=456.63

Description 9:: (S)-N-(1-Phenylpropyl)-3-(4-ethenesulfonyl-piperazin-1-ylmethyl)-2-phenylquinoline4-carboxyamide

2.8 g (6.13 mmol) of the compound of Description 5 and 0.85 g (6.13 mmol) of K₂CO₃ were suspended in 20 ml of CH₂Cl₂ and the mixture was cooled to 0° C. 1 g (6.13 mmol) of 2-chloroethylsulphonylchloride, dissolved in 5 ml of CH₂Cl₂, were added dropwise and the mixture was stirred at room temperature for 2 hours. 10 ml of H₂O were added and the slurry was stirred for additional 10 minutes. The acqueous phase was separated and extracted three times with CH₂Cl₂. The organic phases, collected together were washed with water, dried over Na₂SO₄, filtered and evaporated in vacuo to dryness to yield 1.2 g of a yellow powder. The crude comound was purified by flash chromatography on silicagel (eluent: hexane/EtOAc; 1/1), affording 0.15 g of the title compound.

C₃₂H₃₄N₄O₃S

MW=554.654

IR: (KBr) 3286, 3059, 2933, 1638, 1534, 1492, 1454, 1158, 952, 763, 701 cm-1

### Example 1

3-{4-[4-((S)-2-Methyl-1-phenyl-propylcarbamoyl)-2-phenyl-quinolin-3-ylmethyl]-piperazine-1-sulfonyl}-propionic acid methyl ester

A suspension of 1.2 g (2.5 mmol) of 2-phenyl-3-piperazin-1-ylmethyl-quinoline4-carboxylic acid ((S)-2-methyl-1-phenyl-propyl)-amide (compound of Description 7) in 20 ml of acetonitrile was reacted with 0.513 g (2.75 mmol) of beta carbomethoxyethanesulfonyl chloride and 482 ul (2.75 mmol) of diisopropylethylamine. The reaction mixture was stirred at room temperature for 15 h followed by 3 h at 50° C. The solvent was concentrated and the residue taken up in CH₂Cl₂. The un-reacted starting amine (0.46 g) was filtered off and the solution was washed twice with water, dried over MgSO₄ and concentrated. The crude compound was purified by flash chromatography over 45 g silica gel (eluent: CH₂Cl₂/MeOH: 99/1) to afford 0.48 g of the title compounds as a cream solid.

C₃₅H₄₀N₄O₅S

MW=628.790

M.P.=105-107

### Example 2

3-{4-[4-((S)-2-Methyl-1-phenyl-propylcarbamoyl)-2-phenyl-quinolin-3-ylmethyl]-piperazine-1-sulfonyl}-propionic acid.

A mixture of 0.43 g (6.8 mmol) of 3-{4-[4-((S)-2-methyl-1-phenyl-propylcarbamoyl)-2-phenyl-quinolin-3-ylmethyl]-piperazine1-sulfonyl} -propionic acid methyl ester (compound of Example 1), 4 mml of ethanol and 0.72 ml of 1N aqueous sodium hydroxide were stirred at room temperature for 1 h. The solvent was concentrated and the residue neutralised by a few drops of acetic acid and purified by flash chromatography over 45 g of silicagel (eluent: CH₂Cl₂/MeOH: 95/5). The desired fractions were pooled, concentrated and the residue crystailised in diisoprpyl ether. The compound obtained thereafter being impure the same process (chromatography+crystallisation) was applied again affording now 60 mg of the title compound as light yellow crystals.

C₃₄H₃₈N₄O₅S

MW=614.763

M.P.=150-5° C.

### Example 3

(-)-(S)-N-(1-Phenylpropyl)-3-[(2-N′,N′-diethylamino-1-ethyl)sulphonyl]-2-phenylquinolinle-4-carboxamide

0.1 g (0.18 mmol) of compound of Description 9 and 5 ml (48 mmol) of diethylarnine were dissolved in 10 ml of CH₂Cl₂ and the solution was stirred at room temperature for 15 days.

The solvent was evaporated in vacuo to dryness, the residue was dissolved in AcOEt, washed with water and dried over Na₂SO₄. After concentration of the solvent the residue was purified by flash chromatography (eluent, hexane/AcOEt: 9/1) affording 0.067 g of the title compound as yellow powder.

C₃₆H₄₅N₅O₃S

MW=627.79

M.P.: 93-97° C.

IR: (KBr) 3296, 3059, 2968, 1658, 1531, 1153, 955, 764, 702 cm-1

### Example 4

(S)-N-(1-cyclohexylethyl)-3-(4-methanesulfonylpiperazin-1-ylmethyl)-2-phenylquinoline-4-carboxyamide 0.3 g (0.66 mmol) of (S)-N-(1-cyclohexylethyl)-3-[4-piperazin-1-yl]methyl-2-phenylquinoline-4-carboxamide (compound of description 8), 0.082 g (0.7 mmol) of methanesulfonyl chloride and 0.1 ml (0.7 mnmol) of TEA were dissolved in 20 ml of THF. The solution was stirred at room temperature for 18 hours then the solvent was evaporated in vacuo to dryness. The residue was dissolved in AcOEt. The organic phase was washed with 1N NaOH solution and with water, then dried over Na₂SO₄. After concentration of the solvent the residue was purified by flash chromatography (eluent, hexane/AcOEt: 1/1) affording 0.27 g (78%) of the title compound as white powder.

C₃₀H₃₈N₄O₃S

MW: 534.72

M.P.: 138-140° C.

IR: (nujol) 3283, 1634, 1537, 1455 cm-1

### Example 5

((S)-1-cyclohexylethyl)-3-(4-benzenesulfonylpiperazin-1-ylmethyo)-2-phenylquinoline-4-carboxyamide

0.3 g (0.6 mmol) of (S)-N-(1-cyclohexylethyl)-3-[4-piperazin-1-yl]methyl-2-phenylquinoline-4-carboxamide (compound of description 8), 0.1 ml (0.7 mmol) of benzenesulfonyl chloride and 0.1 ml (0.7 mnmol) of TEA were dissolved in 20 ml of THF. The solution was stirred at room temperature for 18 hours then the solvent was evaporated in vacuo to dryness. The residue was dissolved in AcOEt. The organic phase was washed with 1N NaOH solution and with water, then dried over Na₂SO₄. After concentration of the solvent the residue was purified by flash chromatography (eluent, hexane/AcOEt: 2/1) affording 0.150 g (38%) of the title compound as white powder.

C₃₅H₄₀N₄O₃S

MW: 596.79

M.P.: 208-210° C.

IR: (nujol) 1626, 1553, 1455, 1355 cm-1

TABLE 1

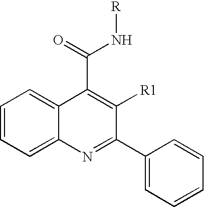

| | | | | | Melting | [a]_{D}²⁰ |
|---|---|---|---|---|---|---|
| | | | Molecular | Molecular | Point | (c = 0.5, |
| Ex. | R | R₁ | Formula | Weight | (° C.) | MeOH) |
| 1 | 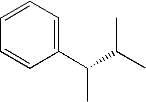 | 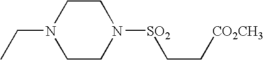 | C₃₅H₄₀N₄O₅S | 628.790 | 105-107 | |
| 2 | 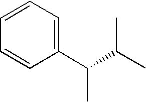 | 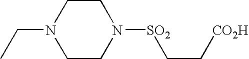 | C₃₄H₃₈N₄O₅S | 614.763 | 150-155 | |
| 3 | 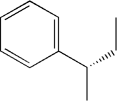 | 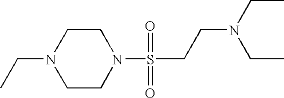 | C₃₆H₄₅N₅O₃S | 627.79 | 93-97 −54.05 (c = 0.26) | |
| 4 | 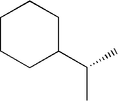 | 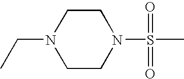 | C₃₀H₃₈N₄O₃S | 534.72 | 138-140 | +13.43 (c = 0.1) |
| 5 | 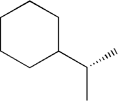 | 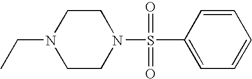 | C₃₅H₄₀N₄O₃S | 596.79 | 208-210 | +13.3 (c = 0.1) |

TABLE 2
¹H NMR data of compounds of Examples of Table 1

| Ex. | ¹H NMR (Solvent) delta ppm |
|---|---|
| 1 | (CDCl₃) δ: 0.94(d, 3H); 1.17(d, 3H); 1.93(m, 4H); 2.24(m, 1H); |
| | 2.73(t, 2H); 2.80(m, 4H); 3.09(t, 2H); 3.53(s, 2H); 3.53(s, 3H); |
| | 5.13(m, 1H); 6.90(d, 1Har); 7.27-7.64(m, 11Har); 7.74(td, 1Har); |
| | 7.89(br, 1H); 8.14(dd, 1Har)ppm |
| 2 | (CDCl3) δ: 0.91(d, 3H); 1.15(d, 3H); 1.91(m, 4H); 2.20(m, 1H); |
| | 2.71(t, 2H); 2.82(m, 4H); 3.10(t, 2H); 3.27(br, 1H); 3.51(s, 2H); |
| | 5.10(m, 1H); 6.88(d, 1Har); 7.25-7.63(m, 11Har); 7.73(t, 1Har); |
| | 7.93(br, 1H); 8.13(d, 1Har)ppm. |
| 3 | (DMSO) (343 K); 8.87(d, 1H); 8.02(d, 1H); 7.76(dd, 1H); |
| | 7.71(m, 1H); 7.60-7.42(m, 8H); 7.38(dd, 2H); 7.28(dd, 1H); |
| | 5.10(dt, 1H); 3.54(s br, 2H); 3.00(m, 2H); 2.81(m, 4H); 2.74(m, 2H); |
| | 2.48(q, 4H); 2.04(m, 4H); 1.98-1.76(m, 2H); 0.98(t, 6H); |
| | 0.97(t, 3H). |
| 4 | (CDCl3): 8.15(d, 1H); 7.98(d, 1H); 7.74(dd, 1H); 7.60(dd, 1H); |
| | 7.52-7.41(m, 5H); 6.56(s br, 1H); 4.26(dt, 1H); 3.76(s, 2H); |
| | 2.98(m, 4H); 2.68(s, 3H); 2.31(m, 4H); 1.98-1.66(m, 5H); |
| | 1.47(m, 1H); 1.35-1.02(m, 5H); 1.29(d, 1H) |
| 5 | (DMSO): 8.20(d br, 1H); 7.99(d, 1H); 7.82(d, 1H); 7.78- |
| | 7.58(m, 7H); 7.41(m, 2H); 7.31(m, 3H); 3.97(dt, 1H); 3.62(s, 2H); |
| | 2.66(m, 4H); 2.18(m, 4H); 1.83-1.60(m, 5H); 1.46(m, 1H); 1.31- |
| | 1.02(m, 5H); 1.13(d, 3H) |

TABLE 3
Mass Spectra data of compounds of Examples of Table 1

| | m/z | m/z |
|---|---|---|
| | (ESI POS; AQA; solvent: | (EI; TSQ 700; |
| | methanol/spray 3 kV/ | source 180 ° C.; |
| Ex. | skimmer: 20 V/probe 135 C) | 70 eV; 200 uA) |
| 1 | | |
| 2 | | |
| 3 | 628 (MH+) | |
| 4 | | 534 (M+); 455; 407; 380; 372; |
| | | 328; 316; 300; 261; 246; 217 |
| 5 | | 442; 372; 370; 328; 316; 300; |
| | | 261; 217; 140 |

TABLE 4
Chemical names of parent compounds of Examples of Table 1
(names generated by Beilstein's Autonom)

| Example | Chemical name |
|---|---|
| 1 | 3-{4-[4-((S)-2-Methyl-1-phenyl-propylcarbamoyl)-2-phenyl- |
| | quinolin-3-ylmethyl]-piperazin-1-sulfonyl}-propionic acid |
| | methyl ester |
| 2 | 3-{4-[4-((S)-2-Methyl-1-phenyl-propylcarbamoyl)-2-phenyl- |
| | quinolin-3-ylmethyl]-piperazin-1-sulfonyl}-propionic acid |
| 3 | (−)-(S)-N-(1-Phenylpropyl)-3-[(2-N′,N′-diethylamino-1- |
| | ethyl)sulphonyl]-2-phenylquinoline-4-carboxamide |
| 4 | (S)-N-(1-cyclohexylethyl)-3-(4-methanesulfonylpiperazin-1- |
| | ylmethyl)-2-phenylquinoline-4-carboxyamide |
| 5 | ((S)-1-cyclohexyl-ethyl)-3-(4-Benzenesulfonylpiperazin-1- |
| | ylmethyl)-2-phenylquinoline-4-carboxyamide |

1. A compound of formula (I) below or a pharmaceutically acceptable salt or hydrate thereof: 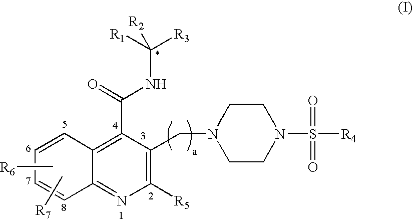
(I)
wherein:
R₁ is H or C₁₋₆ alkyl;
R₂ is aryl or C₃₋₇ cycloalkyl or heteroaryl;
R₃ is H or C₁₋₃ alkyl, optionally substituted by one or more fluorines;
R₄ is R₈R₉;
R₈ is a single bond, C₁₋₆ alkyl, or aryl;
R₉ is H, COO R₁₀, or N R₁₁R₁₂;
R₁₀ is H or C₁₋₆ alkyl;
R₁₁ and R₁₂ are independently selected from H and C₁₋₆ alkyl, or are joined to form a 5-7 membered heterocyclic ring;
R₅ is branched or linear C₁₋₆ alkyl, C₃₋₇ cycloalkyl, C₄₋₇ cycloalkylalkyl, aryl, or a single or fused ring aromatic heterocyclic group;
R₆ represents H or up to three substituents independently selected from the list consisting of: C₁₋₆ alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluorornethyl, acyloxy, amino or mono- or di-C₁₋₆ alkylamino;
R₇ is H or halo;
a is 1-6; and
any of R₂, R₅, R₈, R₁₀, R₁₁ and R₁₂ may optionally be substituted one or more times by halo, hydroxy, amino, cyano, nitro, carboxy or oxo;
subject to the proviso that said compound is not a compound of formula (I) wherein R₇ represents H, R₆ represents H, R₅ represents phenyl, a is 1, and R₁, R₂, R₃ and R₄ are one of the following combinations:

| 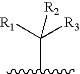 | R₄ |
|---|---|
| 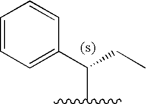 | —NH₂ |
| 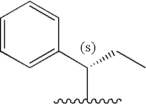 | 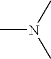 |

2. A compound as claimed in claim 1, wherein R₈ is methyl, ethyl or phenyl.

3. A compound as claimed in claim 1 or claim 2, wherein R₉ is H.

4. A compound as claimed in claim 1 or claim 2, wherein R₉ is COOR₁₀ and R₁₀ is H or methyl or ethyl.

5. A compound as claimed in claim 1 or claim 2, wherein wherein R₉ is NR₁₁R₁₂, and R₁₁ and/or R₁₂ is methyl, ethyl or propyl.

6. A compound as claimed in claim 5, wherein R₁₁ and R₁₂ are the same one of ethyl or propyl.

7. A compound as claimed in claim 1, wherein a is 1, R₆ is H, R₁ is H, R₅ is unsubstituted phenyl, R₇ is hydrogen, and R₂, R₃ and R₄ are selected from the following combinations:

| R₂ | R₃ | R₄ |
|---|---|---|
| phenyl | isopropyl | 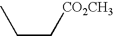 |
| phenyl | isopropyl | 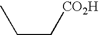 |
| phenyl | ethyl | 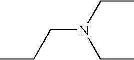 |
| cyclohexyl | methyl | —CH₃ |
| cyclohexyl | methyl | 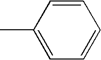 |

8. A process for the preparation of a compound of formula (I) according to any of claims 1-7, or a salt thereof and/or a solvate thereof, which process comprises reacting a compound of formula (II) or an active derivative thereof: 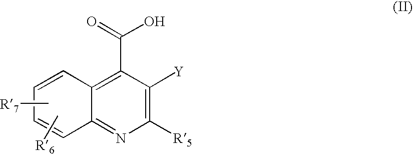
wherein R′₅, R′₆, and R′₇ are R₅, R₆, and R₇ respectively as defmed in relation to formula (I) or a group convertible to R₅, R₆, and R₇ respectively, and Y′ is a group of formula (Y) or a protected form thereof: 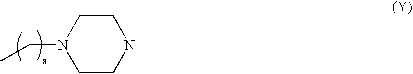
with a compound of formula (III): 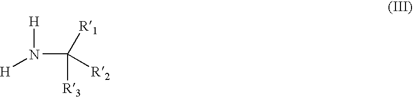
wherein R′₁, R′₂ and R′₃ are R₁, R₂ and R₃ as defined for formula (I) or a group or atom convertible to R₁, R₂ and R₃ respectively; to form a compound of formula (Ib): 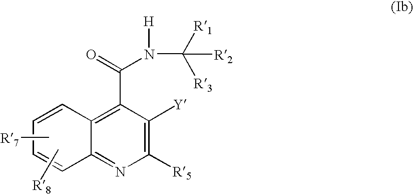
wherein R′₁, R′₂, R′₃, R′₅, R′₆, R′₇ and Y′ are as defined above;
reacting said compound (Ib) with a compound of formula 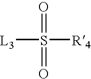
wherein L₃ represents a leaving group for example halogen, preferably chlorine or bromine, and R′₄ represents R₄ or a protected form thereof or a group convertible thereto;
and thereafter carrying out one or more of the following optional steps:
(i) converting any one of R′₁, R′₂, R′₃, R′₄, R′₅, R′₆, and R′₇ to R₁, R₂,R₃, R₄, R₅, R₆, and R₇ respectively as required, to obtain a compound of formula (I);
(ii) converting a compound of formula (I) into another compound of formula (I); and
(iii) preparing a salt of the compound of formula (I) and/or a solvate thereof.

9. A pharmaceutical composition comprising a compound of formula (I) according to any of claims 1-7, or a pharmaceutically acceptable salt or solvate thereof, and a pharmaceutically acceptable carrier.

10. A compound of formula (I) according to any of claims 1-7, or a pharmaceutically acceptable salt or solvate thereof, for use as anactive therapeutic substance.

11. A compound of formula (I) according to any of claims 1-7, or a pharmaceutically acceptable salt or solvate thereof, for the treatment or prophylaxis of the Primary and Secondary Conditions.

12. Use of a compound of formula (I) according to any of claims 1-7, or a pharmaceutically acceptable salt or solvate thereof, in the manufacture of a medicament for the treatment of the Primary and Secondary Conditions.

13. A method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) according to any of claims 1-7 or a pharmaceutically acceptable salt or solvate thereof.
